(19) United States
(12) Patent Application Publication
(10) Pub. No.: US 2011/0294908 A1
(43) Pub. Date: Dec. 1, 2011

(54) FISCHER-TROPSCH SYNTHESIS FE-BASED CATALYST, PROCESS OF PREPARATION AND APPLICATION THEREOF

(76) Inventors: Baoshan Wu, Shanxi (CN); Yong Yang, Shanxi (CN); Yongwang Li, Shanxi (CN); Hongwei Xiang, Shanxi (CN)

(21) Appl. No.: 13/148,209

(22) PCT Filed: Feb. 9, 2010
(86) PCT No.: PCT/CN2010/070569

§ 371 Date: Aug. 5, 2011

(30) Foreign Application Priority Data
Feb. 20, 2009 (CN) 200910005362.9

(51) Int. Cl. C07C 1/04; B01J 21/08

(52) U.S. Cl. 518/713; 502/243; 502/245

(57) ABSTRACT

This invention relates to a Fe-based catalyst for Fischer-Tropsch synthesis, preparation method and application thereof. The catalyst contains Fe, oxide(s) of IB group metal Cu and/or Ag as a reducing promoter, IA group metal Li, Na, K or Rb as an electron promoter, VIII group noble metal Ru, Rh, Pd or Pt as a hydrogenation promoter and SiO₂ as a structure promoter. The preparation method comprises the following steps: preparing a solution of Fe salt; co-precipitating the solution rapidly with an alkaline compound, then washing and pulping again; and adding a solution of IB group metal salt as a reducing promoter, a IA group metal salt solution and silica sol, or adding a solution of IB group metal salt as a reducing promoter and a silicate of IA group metal; then molding by spray-drying, impregnating in a solution of VIII group noble metal salt, and drying and roasting to obtain the catalyst. The catalyst is suitable for producing hydrocarbons by a low temperature Fischer-Tropsch synthesis process. The process has a high yield of heavy hydrocarbons and a low selectivity to methane and significantly reduces the selectivity to olefins.

### FIELD OF THE INVENTION

The present invention relates to a carbon monoxide (CO) hydrogenation catalyst and preparation method thereof, more specifically, the invention relates to a Fe-based catalyst used in Fischer-Tropsch synthesis, preparation method thereof and use of the catalyst for producing hydrocarbons by Fischer-Tropsch synthesis.

### BACKGROUND OF THE INVENTION

Fischer-Tropsch synthesis as a method for producing liquid fuels from syngas (CO+H₂) is invented in Germany at 1920s and gradually turned to industrial applications, wherein the syngas is derived from coal, natural gas, coal-bed gas and other biomass containing carbon. The main active metals of Fischer-Tropsch synthesis catalyst are VIII group metals, wherein only Fe, Co, Ni and Ru have sufficiently high activity of CO hydrogenation. They have important value for the application of Fischer-Tropsch synthesis, wherein Ru has the highest catalytic activity, and can achieve a fairly high yield of heavy hydrocarbons even CO hydrogenation is reacted at 150° C., but the low reserves and high price limit its large-scale industrial applications. The catalytic activity of Ni-based catalyst is also high, but has two main drawbacks: firstly, strong hydrogenation ability of Ni-based catalyst results in more CH₄ generated than that of Fe-based and Co-based catalysts in Fischer-Tropsch synthesis products; secondly, under a typical industrial operating condition of Fischer-Tropsch synthesis, Ni can be easily converted into a volatile metal carbonyl compound, which results in a continuous loss of nickel. Therefore, only Fe and Co have potential industrial applications value.

Co catalyst is the earliest Fischer-Tropsch synthesis catalyst of industrial production, which is characterized by higher single-pass conversion rate, longer life-time and higher straight-chain hydrocarbons yield at a relatively low temperature. Despite Co catalyst has a higher cost, it is more suitable for operating on a fixed bed reactor, easily to be recovered and regenerated. In addition, due to low water-gas shift (WGS) activity, Co catalyst is preferable for the FTS of syngas with high H₂/CO ratio derived from natural gas. Co catalyst has been studied and reported in a lot of literatures and patents, wherein Co catalyst produced by Shell Company has been successfully achieved industrial applications of several years.

The features of Fe catalyst are as follows: low prices, allowing a wide range of operating temperature (220-350° C.); the selectivity of methane can be kept relatively low even at a very high temperature; product selectivity can be modulated; In addition, due to high water-gas shift reaction activity, Fe catalyst is preferable for the conversion of coal-based syngas with low H₂/CO ratio (H₂/CO=0.5-0.7). In 1950s, the Sasol Company in South Africa has successfully used Fe catalyst for the industrialization of coal-based synthetic liquid fuels. The Fe catalysts which can be used in Fischer-Tropsch synthesis include fused Fe catalyst and precipitated Fe catalyst. Up till now, the preparation methods of precipitated Fe catalysts have been reported in a considerable number of literatures.

In U.S. Pat. No. 4,617,288 and U.S. Pat. No. 4,686,313, Mobil (USA) disclosed a continuous co-precipitation method to prepare a Fe—Cu—K FTS catalyst of a low nitrogen content, specifically, the method is as follows: continuously co-precipitating a mixed solution containing ferric nitrate and copper nitrate according to the stoichiometric ratio with ammonia of an appropriate concentration under a pH of 6.6-6.8 and a temperature ranging from 80° C. to 90° C., vacuum filtering, washing, adding a certain amount of potassium carbonate solution, pulping, drying and roasting at 300° C. to obtain the catalyst of a low nitrogen content. This method is suitable for continuous industrial production. In U.S. Pat. No. 4,994,428, Mobil also disclosed a method for preparing co-precipitated Fe—Cu—K catalyst and a method for treating this catalyst with water vapor, which makes the content of C₅⁺ in the hydrocarbon product 90% or more.

Sasol Technology Ltd in U.S. Pat. No. 6,844,370 also disclosed a preparation method for a precipitated Fe—Cu—K FTS catalyst without binders, which is suitable for hydrocarbon synthesis in high temperature fluidized bed.

In U.S. Pat. No. 5,504,118 and CN 1113905A, Rentech (USA) disclosed a preparation method of Fe-based FTS catalyst used in slurry bed reactor, specifically, the method is as follows: dissolving metals iron and copper with nitric acid to obtain the nitrates, followed by adding ammonia into the hot mixed solution of the nitrates, and controlling pH value at 7.4 to obtain a slurry of the precipitate, then filtering, washing, adding a potassium carbonate solution, pulping to the weight percent of the catalyst in the slurry of about 8-12%, spray-drying by using a spray-dryer and finally roasting the catalyst in the air at 315° C. to obtain the catalyst product. Chevron (USA) in U.S. Pat. No. 6,787,577 disclosed a method for catalyst preparation using co-precipitation of organosilicon and iron salt and its application in FTS. The co-precipitated slurry is treated by washing, filtering, drying, and then impregnated with a K₂O₃ and Cu(NO₃)₂ aqueous solution according to a certain proportion, thus a Fe—Cu—K—Si catalyst is obtained. The Fe—Cu—K—Si catalyst is characterized by high olefins yield of C₂-C₄ and C₅-C₁₁.

Exxon (USA) in U.S. Pat. No. 5,100,556 disclosed a preparation method of precipitated Fe—Zn—Cu—K catalyst and its application in FTS. This method includes: co-precipitating the Fe/Zn mixed nitrates with ammonia at a pH of about 6.5, washing the filter cake obtained and filtering, drying, then impregnating with a K₂CO₃ solution according to the proportion, drying, and then impregnating with a Cu(NO₃)₂ solution, drying and roasting. This method improved the activity and stability of the catalyst, and facilitated the production of α-olefins.

A paper by Burkur et al from Texas A & M University USA [Ind. Eng. Chem. Res., 1990, 29, p1588-1599] disclosed a preparation method of Fe/Cu/K/SiO₂ catalyst, specifically, the method is as follows: continuously co-precipitating a mixed solution containing iron nitrate and copper nitrate with ammonia at 82° C.; washing the precipitate thoroughly and filtering; adding a certain amount of K₂SiO₃ solution, pulping again, adjusting the pH to 6 or lower, vacuum drying, then impregnating with a KHCO₃ solution, drying and roasting at 300° C. for 5 h to obtain the catalyst. The paper [Ind. Eng. Chem. Res., 1999, 38, p3270-3275] disclosed a 100Fe/3Cu/4K/16SiO₂ (by weight) catalyst was used in a slurry bed reactor, and it was found that the catalyst had a high activity to syngas with low ratio of H₂/CO and a high selectivity to C₅⁺ and light olefins.

The catalysts above mentioned have different characteristics at their composition, preparation methods, applications and so on according to the different syngas source and target products, but part of catalysts have not entered into the substantive industrial application stage.

### DETAILED DESCRIPTION OF THE INVENTION

The objective of the present invention is to provide a CO hydrogenation catalyst, specifically, the catalyst is a Fe-based FTS catalyst with the main component of Fe. The catalyst also contains: oxide(s) of IB group metal Cu and/or Ag as a reducing promoter; at least one oxide of IA group metal M as an electron promoter, wherein the IA group metal M is selected from Li, Na, K or Rb; at least one VIII group noble metal M′ as a hydrogenation promoter, the VIII group noble metal M′ is selected from Ru, Rh, Pd or Pt; and SiO₂ as a structure promoter. The main component Fe content in the catalyst is 30 wt %-70 wt %, preferably 40 wt %-65 wt %, more preferably 45 wt %-60 wt %.

In the Fe-based catalyst according to the present invention, the Fe exists in the form of complete oxide (i.e., the highest oxidation valency (Fe(III)). Hereinafter, the metal component is calculated based on the metal element and the structure promoter is calculated based on the oxide.

The Fe-based catalyst according to the present invention contains at least one oxide of IB group metal as a reducing promoter, preferably oxide(s) of Cu and/or Ag. The weight ratio of Fe/Cu is 100/0-20, preferably 100/0-12, more preferably 100/0-8; The weight ratio of Fe/Ag is 100/0-10, preferably 100/0-5, more preferably 100/0-2.

The Fe-based catalyst according to the present invention contains at least one oxide of IA group metal M as an electron promoter, the IA group metal M is selected from Li, Na, K or Rb, preferably Li, K or Na, more preferably K or Li; the weight ratio of Fe to the electron promoter (expressed as Fe/M) is 100/0.05-15, preferably 100/0.1-10, more preferably 100/0.5-8.

The Fe-based catalyst according to the present invention further contains SiO₂ as a structure promoter, and the weight ratio of Fe/SiO₂ is 100/0.1-50, preferably 100/1-45, more preferably 100/5-35.

The Fe-based catalyst according to the present invention also contains a small amount of VIII group noble metal M′ as a hydrogenation promoter, and the VIII group noble metal M′ is selected from Ru, Rh, Pd or Pt, preferably Ru or Pt. The weight ratio of Fe (the main component of the catalyst) to the noble metal M′ (expressed as Fe/M′) is 100/0.001-5, preferably 100/0.005-1, more preferably 100/0.01-0.5.

In the Fe-based catalyst according to the present invention, preferably the IA group metal M as the electron promoter is the oxide of K or Li, and the VIII group metal M′ as the hydrogenation promoter is Ru or Pt.

Preferably, the Fe-based catalyst according to the present invention is composed of Fe, Cu and/or Ag as a reducing promoter, an electron promoter, a hydrogenation promoter and a structure promoter; wherein the ratio of weight parts of each component expressed as Fe:Cu:Ag: the electron promoter:the hydrogenation promoter:the structure promoter is equal to 100:0-20:0-10:0.05-15:0.001-5:0.1-50; preferably, the weight ratio is 100:0-12:0-5:0.1-10:0.005-1:1-45, more preferably, the weight ratio is 100:0-8:0-2:0.5-8:0.01-0.5:5-35, under the condition that the contents of Cu and Ag cannot be zero simultaneously.

The preferable components of the Fe-based catalyst according to the present invention is, for example, Fe/Cu/Li/Ru/SiO₂, Fe/Ag/K/Pt/SiO₂, or Fe/Cu/K/Pt/SiO₂ In the Fe-based catalyst of the present invention, a small amount of VIII group noble metal is added as the hydrogenation promoter. According to the common knowledge in this field, the hydrogenation activity of catalyst increase after adding the noble metal. The increase in the hydrogenation activity of catalyst should be extremely limited because the amount of the noble metal added in the catalyst is small; but by the selection and optimization of other promoters and the amount thereof, the synergistic effects of therebetween allow the catalyst of the present invention not only to achieve improved hydrogenation activity in a greater extent, but also achieve a very good modulation effect in the product selectivity.

Therefore, the catalyst according to the present invention is suitable for Fischer-Tropsch synthesis, especially suitable for low temperature Fischer-Tropsch synthesis, and more especially, suitable for low temperature Fischer-Tropsch synthesis in the slurry bed reactor.

The other objective of the present invention is to provide a preparation method of Fe-based catalyst above mentioned. This method is characterized by: simple preparation process and a variety of Fe sources for use, and adding a small amount of noble metal promoter without altering the total production costs which not only improved the activity of the catalyst, but also could adjust the selectivity of the products.

The preparation method of the above mentioned Fe-based catalyst in the present invention includes the following steps:

(1) preparing a solution of Fe salt;
(2) co-precipitating the solution of Fe salt with an alkaline precipitant to obtain a precipitate;
(3) pulping the precipitate again after being washed, and according to the required proportion, adding a solution of IB group metal Cu and/or Ag salt, a solution of IA group metal M salt and a structure promoter SiO₂ to obtain a mixed slurry, wherein the structure promoter SiO₂ is silica sol or a silicate of IA group metal M; and
(4) then molding by spray-drying the mixed slurry to obtain a molded catalyst, according to the required proportion, isometric impregnating the molded catalyst in a solution of at least one kind of VIII group noble metal M′ salt, then drying and roasting to obtain the catalyst.

In the above mentioned method, wherein adding the solution of IB group metal Cu and/or Ag salt in the step (3) can be performed in the step (1), that is, the solution of IB group metal Cu and/or Ag salt is added into the Fe salt solution.

In the above mentioned method, wherein introducing the structure promoter SiO₂ in the step (3) can be performed in the step (1), or part of the structure promoter is added in the steps (1) and (3) respectively, and can adjust the proportion added in the two steps if necessary.

Preferably, the process for adding the solution of IB group metal Cu and/or Ag salt can be freely chosen and adjusted; also, the process for introducing the structure promoter SiO₂ can be freely chosen and adjusted, for example, part of the structure promoter is added in the steps (1) and (3) respectively, and can freely adjust the proportion added in each steps.

In the method of the present invention, every component of the catalyst such as the reducing promoter, the electron promoter and the hydrogenation promoter is chosen as mentioned above, wherein the addition amount “according to the required proportion” is the content ratio of each above-mentioned component.

In the method above mentioned in the present invention, the IA group metal M as the electron promoter is selected from Li, Na, K or Rb, preferably K or Li, more preferably K; the VIII group noble metal M′ as the hydrogenation promoter is selected from Ru, Rh, Pd or Pt, preferably Ru or Pt.

In the method above mentioned in the present invention, the solution of Fe salt used in the step (1) is the aqueous solution of ferric (Fe³⁺) salt, for example, the ferric (Fe³⁺) salt is ferric nitrate or ferric sulfate; in the case that the solution of Fe salt is the solution of ferric nitrate, the solution can be obtained through dissolving industrial ferric nitrate; or through dissolving scrap iron with nitric acid; in the case that the solution of Fe salt in the step (1) is the solution of ferric sulfate, the solution can be obtained through dissolving industrial polymeric ferric sulfate, or through dissolving industrial ferrous sulfate followed by oxidation; the concentration of solution of Fe salt is 0.5-10 wt %, preferably 1-5 mol/L.

In the method above mentioned in the present invention, the co-precipitation technique known in this field is used in the step (2), preferably co-precipitation by coflowing technique, the alkaline precipitant used for co-precipitation is selected from Na₂CO₃, ammonia, K₂CO₃, (NH₄)₂CO₃, (NH₄)HCO₃ and so on, preferably Na₂CO₃, ammonia or (NH₄)₂CO₃; a molar concentration of the alkaline precipitant aqueous solution is 1-6 mol/L, preferably 1.5-4.5 mol/L; Usually, the amount of the alkaline precipitant used in the step (2) is prepared according to the stoichiometry, but preferably slightly excessive than the stoichiometry; wherein the precipitation temperature in the step (2) is 20-95°, and the pH of the slurry for precipitation is from 5 to 10, and the precipitation time is 5-60 min; preferably the precipitation temperature is 40-90° C., pH is from 6 to 10; more preferably, the precipitation temperature is 55-85° C., pH is from 6 to 9 and precipitation time is 10-30 min; and wherein the aging time is 5 min-2h, preferably 5-30 min.

In the method above mentioned in the present invention, wherein the water used preferably is deionized water, such as distilled water. By the method according to the present invention, the water amount consumed in the preparation is greatly saved. For example, when using ammonia or (NH₄)₂CO₃ as the precipitant in the step (2), the water can be saved by 50%, compared with the traditional process.

In the method above mentioned in the present invention, the solution of Cu salt in the step (3) is the solution of copper nitrate or copper sulfate with a concentration of 1-4 mol/L, preferably 1.5-3.0 mol/L; wherein the solution of Ag⁺ salt is silver nitrate solution with a concentration of 0.1-3 mol/L, preferably 0.5-1.5 mol/L.

In the method above mentioned in the present invention, the structure promoter SiO₂ in the step (3) is silica sol or silicate of IA group metal M, wherein the silica sol is acidic silica sol or basic silica sol, and the concentration of SiO₂ in the structural promoter is 5-50 wt %, preferably 15-40 wt %.

In the case that the SiO₂ added into the slurry of the precipitate in the step (3) is silicate of IA group metal M, the silicate is the silicate solution prepared in advance from SiO₂ and IA group metal M according to the required proportion, and the silicate of IA group metal M is prepared from industrial M sodium silicate and silica sol; in the solution of the silicate of IA group metal M, the molar ratio of SiO₂/M₂O is 1-10, preferably the molar ratio of SiO₂/M₂O is 2-5.

In the method above mentioned in the present invention, the IA group metal M salt solution in the step (3) can be carbonate, acid carbonate, nitrate or acetate solution of the metal M and so on, preferably carbonate solution or acetate solution; the salt concentration is 0.5-25 wt %, preferably 10-20 wt %.

In the method above mentioned in the present invention, if the SiO₂ added is silicate of IA group metal M, wherein the contents of the IA group metal M and SiO₂ should be calculated into the electron promoter and the structure promoter, respectively; in addition, followed by the addition process in the step (3), the obtained mixture is required to be mixed well and high speed cut to prepare catalyst slurry.

In the method above mentioned in the present invention, the step (4) includes: firstly, molding by spray-drying the above catalyst slurry, selecting the particles with appropriate diameters, then impregnating the above particles in the solution of the VIII group noble metal M′ salt. The impregnating method can use the routine technique in the art, such as isometric impregnating method, preferably, the solution of the VIII group noble metal M′ salt is nitrate solution, and the concentration of the salt solution used can be determined according to the proportion of Fe/M′ in the prepared catalyst and the amount of the solution in the isometric impregnation.

In the method above mentioned in the present invention, the drying temperature in the step (4) is 60-120° C., preferably 80-100° C.; roasting temperature is 200-600° C., preferably 300-550° C.

In the method above mentioned in the present invention, where the metal salt solution is concerned, the solution is the aqueous solution of the salt.

Preferably, the method of preparing the Fe-based catalyst according to the present invention includes the following steps:

(1) Preparing a solution of ferric nitrate or ferric sulfate with a concentration of 1-5 mol/L;
(2) Preparing an aqueous solution of an alkaline precipitant with a concentration of 1.5-4.5 mol/L, the amount of the solution is slightly excessive than the stoichiometry;
precipitating by coflowing the solutions of (1) and (2) at a temperature of 55-85° C. to obtain a slurry of the precipitate with a pH of 6-9 and a precipitation time of 10-30 min; standing still and aging the slurry of the precipitate obtained, vacuum filtering, and washing;
(3) adding water into the washed precipitate and pulping, then adding solution of Cu salt with a concentration of 1.5-3.0 mol/L and/or Ag salt with a concentration of 0.5-1.5 mol/L, silica sol or silicate of IA group metal M with a SiO₂ concentration of 15-40 wt % and a solution of IA group metal M salt with a concentration of 10-20 wt %, and stirring well to obtain the catalyst slurry; and
(4) molding by spray-drying the above catalyst slurry, and selecting the particles of 50-100 pm; according to the required proportion, isometric impregnating the above particles in a solution of VIII group noble metal M′ salt, then drying and roasting to obtain the finished catalyst;

Wherein adding the solution of Cu and/or Ag salt in the step (3) can be performed in the step (1); and/or introducing the structure promoter in the step (3) can be changed to be performed in the step (1), or part of the structure promoter is added in the steps (1) and (3) respectively, and the proportion added in the two steps is adjusted if necessary.

In comparison to the prior art, the Fe-based catalyst according to the present invention and the preparation method thereof have the following advantages:

(1) A small amount of noble metal is added as hydrogenation promoter in the Fe-based catalyst of the present invention, although the addition quantity of the noble metal is small and generally added according to the ratio of Fe/M′=100/0.01-0.5, the improvement in the activity and modulation effect on selectivity for the product of the Fischer-Tropsch synthesis catalyst is highly significant; the olefins selectivity in the heavy hydrocarbons can be controlled through modulating the ratio of the addition amount of the noble metal promoter to other promoters.
(2) In the preparation method of Fe-based catalyst according to the present invention, the methods for adding the reducing promoter are more flexible, in which the reducing promoter can be co-precipitated with the main metal component or added into the slurry before molding the catalyst to avoid the loss of effective components of IB group metals in the preparation process; moreover, due to the excellent features of the reducing promoter and the electron promoter, Ag can bring beneficial effects on adjusting the products selectivity of Fischer-Tropsch synthesis reaction catalyst.
(3) In the preparation method of Fe-based catalyst according to the present invention, the structure promoter (SiO₂) can be added to co-precipitate with the main metal component, also can be added into the slurry before molding the catalyst or added in the two steps above mentioned in different proportions, respectively. The adding methods are flexible and by choosing different adding methods and adjusting the adding proportions in the two steps, the catalyst can be ensured enough Fischer-Tropsch synthesis reaction activity, and the strength of the catalyst can be improved simultaneously as far as possible to ensure its physical stability and chemical stability.
(4) The feature of Fe-based catalyst according to the present invention is that the Fe source can be a variety of raw materials, and the precipitant also can be selected according to practical conditions. In the case that the total cost of catalyst does not increase, different raw materials can be chosen. For example, when using iron nitrate with a relatively high price as the raw material, ammonia or ammonium carbonate can be selected as the precipitant, which can reduce the cost by saving distilled water used for washing.

The other objective of the present invention is to provide a Fischer-Tropsch synthesis method for producing hydrocarbons, characterized in that the Fe-based catalyst mentioned above is used in the method.

Wherein the Fischer-Tropsch synthesis method can be a high temperature Fischer-Tropsch synthesis reaction, also can be a low temperature Fischer-Tropsch synthesis reaction, preferably the low temperature Fischer-Tropsch synthesis reaction (LTFT); the Fischer-Tropsch synthesis reaction can be conducted in the routine reactors for Fischer-Tropsch synthesis, such as fixed bed reactor, suspension bed reactor or slurry bed reactor and the like, preferably the Fischer-Tropsch synthesis reaction is conducted in the slurry bed reactor, more preferably, the slurry bed reactor is the slurry bed reactor with tail gas cycled.

In the above mentioned Fischer-Tropsch synthesis reaction, due to using the catalyst according to the present invention, high space-time yield of effective hydrocarbons product can be ensured, and the CH₄ selectivity can be controlled at a very low level, generally 4 wt % or less, and the olefins selectivity in heavy hydrocarbons is lower; for example, in the prior art, the C₅-C₁₁ olefins selectivity of Fe/Cu/K catalyst mostly is about 80 wt %, whereas in the present invention, the olefins selectivity of the Fe/Cu/K catalyst containing Pt content of Fe/Pt=100/0.01 is decreased to 60 wt % or less.

The above mentioned objective of the present invention is achieved in this way: the Fischer-Tropsch synthesis reaction is carried out in the slurry bed reactor with tail gas cycled, firstly, pretreating the Fe-based catalyst according to the present invention in the reactor, i.e. achieving the on-line reduction of the catalyst; then under the action of the pretreated catalyst, the high efficient Fischer-Tropsch synthesis reaction is conducted according to the low temperature Fischer-Tropsch synthesis reaction operating conditions.

Therefore, the reaction process for producing hydrocarbons involved in the present invention includes the pretreatment process of the catalyst above mentioned and subsequent Fischer-Tropsch synthesis process. The pretreatment process is as follows: exposing the catalyst above mentioned to a reducing atmosphere having appropriate temperature, pressure and space velocity, then on-line reducing in the slurry bed reactor with tail gas cycled for an appropriate time, and using the reduced catalyst to conduct the Fischer-Tropsch synthesis reaction to produce hydrocarbons under routine LTFT operating conditions.

The pretreatment process involved in the Fischer-Tropsch synthesis method of the present invention includes the following steps: mixing the catalyst and molten Fiseher-Tropsch wax well to obtain a slurry, then loading into a slurry bed reactor with tail gas cycled, passing the inactive gas for purging, and then importing the reducing gas; adjusting the pressure of the reactor to the reduction pressure of 0.1-5 MPa, the space velocity of reducing gas to 0.5-5.0 NL/g-cat/h, gradually warming to the reduction temperature of 180-300° C., reducing for 2-48 h, then importing the reducing gas containing inactive gas in which the volume percentage of the inactive gas is 1%-20%, the rest is syngas with ratio of H₂/CO of 0.5-40; after finishing the process, switching the operating conditions to routine operating conditions of the low-temperature Fischer-Tropsch synthesis reaction to produce hydrocarbons by Fischer-Tropsch synthesis.

In the method above mentioned in the present invention, wherein the routine operating conditions of low temperature Fischer-Tropsch synthesis reaction are as follows: the reaction temperature is 210-290° C., the reaction pressure is 0.5-5 MPa, the space velocity of the entrance raw material syngas is 0.5-5.0 NL/g-cat/h and the H₂/CO of the entrance raw material syngas is 0.5-3.5; preferably, the operating conditions of the Fischer-Tropsch synthesis are as follows: the reaction temperature is 220-270° C., the reaction pressure is 1-4 MPa, the space velocity of the entrance raw material syngas is 1.0-4.0 NL/g-cat/h and the H₂/CO of the entrance raw material syngas is 0.7-2.5.

In the pretreatment process above mentioned, the cycling ratio of the tail gas is 1-3; the reducing gas is pure H₂, pure CO or syngas; in the case that the reducing gas is syngas, the hydrogen-to-carbon ratio of the syngas is 0.01-99, preferably 0.1-50, more preferably 2-50.

In the pretreatment process above mentioned, the inactive gas used is N₂ or Ar; in the inactive gas-containing reducing gas used, the volume percent of the inactive gas in the reducing gas is 5%-15%.

In the pretreatment process above mentioned, preferably the reduction temperature is 210-280° C.; preferably the reduction pressure is 0.25-4 MPa; preferably the space velocity of the reducing gas is 1.0-4.0 NL/g-cat/h.

In comparison to the prior art, the low temperature Fischer-Tropsch synthesis method using the Fe-based catalyst of the present invention has the following advantages:

(1) The pretreatment process of the Fischer-Tropsch synthesis method according to the present invention can achieve on-line reduction of catalyst without additional reduction reactor, and the reducing conditions are mild. The switching process of reduction conditions and reaction conditions are protected with inactive gas to avoid changing catalyst physical properties caused by drastic changes of the conditions.
(2) In the method above mentioned in the present invention, the Fischer-Tropsch synthesis process allows the catalyst to operate at higher space velocity to obtain the ideal space-time yield of hydrocarbon products.
(3) The Fe-based catalyst in the present invention has a very high Fischer-Tropsch synthesis reaction activity, and high selectivity for hydrocarbons with five carbons or more and low carbon olefins, the methane selectivity can be controlled at a very low level, and the selectivity of the olefins in heavy hydrocarbons is lower, and it is suitable for synthesizing diesel, gasoline and paraffin products from coal-based syngas in the slurry bed reactor.

### EXAMPLES

The specific preparation examples of Fe-based catalyst and the specific reaction examples of Fischer-Tropsch synthesis are provided in detail to explain the present invention, and the provided examples are only for illustrating the present invention without limiting the protection scope of the present invention by any means.

### Example 1

### Preparation of Catalyst

Dissolve 1.0 T of FeSO₄.7H₂O in 3.0 m³ of deionized water, and followed by adding 70 L of 3 mol/L H₂SO₄ solution. Then add 450 L of 15% H₂O₂ aqueous solution into the mixed solution and oxidize for 2 hours under intense stirring at 20° C. Dissolve 47 kg of CuSO₄.5H₂O in 100 L of deionized water, then add into the ferric sulfate solution prepared above. Dissolve 0.7 T of Na₂CO₃ in 2.5 m³ of deionized water to obtain a sodium carbonate solution. Heat the two solutions obtained to a temperature of 70° C. separately and precipitate by coflowing, keep the temperature of a precipitation tank at 75° C., the pH of the mixture solution at 7-7.5 and accomplish the co-precipitation process by mixing in 15 min, then stand still and aging for 30 min. Filter and wash with deionized water until no SO₄²⁻ is detected to obtain a filter cake. Pulping the filter cake again after adding water, to which an appropriate amount of Li₂CO₃ aqueous solution is added according to a ratio of Fe/Li=100/2, then stir thoroughly, add an appropriate amount of alkaline silica sol (the content of SiO₂ in the alkaline silica sol is 40 wt %) according to the ratio of Fe/SiO₂=100/30, and high-speed cut after violently stirring to obtain a slurry. Then spray-dry the slurry to obtain a dried spherical catalyst, take a required amount of the dried spherical catalyst to be isometric impregnated with a ruthenium nitrate solution according to the ratio of Fe/Ru=100/0.2, after full impregnation, dry with a water bath of 85° C., and then roast at 400° C. for 6 h to obtain a Fe-based catalyst with the weight ratio in the catalyst of Fe/Cu/Li/SiO₂/Ru=100:6:2:30:0.2, and the catalyst is recorded as A.

### Example 2

### Preparation of Catalyst

Dissolve 1.0 T of Fe(NO₃)₃.9H₂O in 2.0 m³ of deionized water, and prepare 2.0 m³ of 10 wt % ammonia with liquid ammonia and water. Heat the two solutions obtained to a temperature of 40° C. separately and precipitate by coflowing, keep the temperature of a precipitation tank at 65° C., the pH of the mixture solution at 8.5-9 and accomplish the co-precipitation process by mixing in 10 min, then stand still and aging for 20 min. Filter and wash with an appropriate amount of deionized water to obtain a filter cake. Pulping the filter cake again after adding water, to which an appropriate amount of a silver nitrate aqueous solution is added according to a ratio of Fe/Ag=100/0.5 and stir thoroughly; add a potassium silicate solution containing SiO₂ of 25 wt % with a modulus of 3.3 according to a ratio of Fe/K/SiO₂=100/6/16, high-speed cut after stirring well to obtain a slurry, then spray-dry the slurry to obtain a dried spherical catalyst. Take the required amount of the dried spherical catalyst to be isometric impregnated with a platinum nitrate solution according to the ratio of Fe/Pt=100/0.01, after full impregnation, dry with a water bath of 90° C., and then roast at 500° C. for 3 h to obtain a Fe-based catalyst with the weight ratio in the catalyst of Fe/Ag/K/SiO₂/Pt=100:0.5:6:16:0.01, and the catalyst is recorded as B.

### Example 3

### Preparation of Catalyst

0.5 T of Fe ingot and 10 kg of scrap copper are reacted with an appropriate amount of a 20 wt % nitric acid solution, and the tail gas is firstly sprayed and absorbed with deionized water, then absorbed with a sodium carbonate solution. Introduce 4.0 m³ of a mixture solution of iron nitrate and copper nitrate prepared into an acid tank; prepare 4.0 m³ of a saturated ammonium carbonate solution with water in a base tank. Heat the two solutions obtained to a temperature of 80° C. separately and precipitate by coflowing, keep the temperature of a precipitation tank at 85° C., the pH of the mixture solution at 7.0-7.5 and accomplish the co-precipitation process by mixing in 40 min, then stand still and aging for 10 min. Filter and wash with an appropriate amount deionized water to obtain a filter cake. Pulping the filter cake again after adding water, to which an appropriate amount of a potassium acetate aqueous solution is added according to a ratio of Fe/K=100/3 and stir thoroughly, then add an appropriate amount of acidic silica sol containing 20 wt % SiO₂ according to a ratio of Fe/SiO₂=100/10, high-speed cut after stirring to obtain a slurry, then spray-dry the slurry to obtain a dried spherical catalyst.

Take the required amount of the dried spherical catalyst to be isometric impregnated with a platinum nitrate solution according to a ratio of Fe/Pt=100/0.1, after full impregnation, dry with a water bath of 85° C., and roast at 450° C. for 5 h to obtain the Fe-based catalyst with the weight ratio in the catalyst of Fe/Cu/K/SiO₂/Pt=100:2:3:10:0.1, and the catalyst is recorded as C.

### Example 4

### Pretreatment Process of Catalyst

Mix 25 g of Catalyst A and 400 mL of molten Fiseher-Tropsch wax well to make into a slurry, then load into a 1 L of slurry bed reactor with tail gas cycled, wherein the cycling ratio is 3, pass N₂ for purging, then import pure H₂ as a reducing gas, and adjust a pressure of the reactor to the reduction pressure 0.101 MPa. A space velocity of the reducing gas is 1.0 NL/g-cat/h, and gradually warmed to a reduction temperature of 220° C. to reduce for 8 h, then the mixed gas of N₂/(CO+H₂) is imported, wherein the H₂/CO of the syngas (CO+H₂) is 2, and the volume percentage of N₂ in the mixed gas is 10%. In the process, operating conditions are gradually switched to the following routine operating conditions for the low temperature Fischer-Tropsch (LTFT) synthesis reaction and begin the process of producing hydrocarbons: the reaction temperature is 240° C., the reaction pressure is 2.0 MPa, the space velocity of the entrance raw materials syngas is 2.5 NL/g-cat/h and the ratio of H₂/CO of the entrance raw materials syngas is 2.

### Example 5

### Pretreatment Process of Catalyst

Mix 10 g of Catalyst B and 300 g of molten Fiseher-Tropsch wax well to make into a slurry, then load into a 1 L of slurry bed reactor with tail gas cycled, wherein the cycling ratio is 2, pass argon gas for purging, then import the syngas (H₂/CO=10) as the reducing gas, and adjust the pressure of the reactor to the reduction pressure of 2.5 MPa. The space velocity of the reducing gas is 4.0 NL/g-cat/h, gradually warmed to the reduction temperature of 250° C. to reduce for 24 h, then a mixed gas of Ar/(CO+H₂) is imported, wherein the H₂/CO of the syngas (CO+H₂) is 30, and the volume percentage of Ar in the mixed gas is 15%. In the process, the operating conditions are switched to the following routine operating conditions for LIFT synthesis and begin the process of producing hydrocarbons: the reaction temperature is 260° C., the reaction pressure is 3.0 MPa, the space velocity of the entrance raw materials syngas is 2.0 NL/g-cat/h and the ratio of H₂/CO of the entrance raw materials syngas is 1.2.

### Example 6

### Fischer-Tropsch Synthesis Reaction Process for Producing Hydrocarbons

Mix 20 g of Catalyst C and 400 mL of molten Fiseher-Tropsch wax well to make into a slurry, then load into a 1 L of slurry bed reactor with tail gas cycled and the cycling ratio is 2, pass N₂ for purging, then import pure CO as the reducing gas, and adjust the pressure of the reactor to the reduction pressure of 3.0 MPa. The space velocity of the reducing gas is 2.0 NL/g-cat/h, gradually warmed to a reduction temperature of 270° C. to reduce for 36 h, then a mixed gas of N₂/(CO+H₂) is imported, wherein the H₂/CO of syngas (CO+H₂) is 10 and the volume percentage of N₂ in the mixed gas is 20%. In the process, the operating conditions are switched to the following routine operating conditions for LTFT synthesis and begin the process of producing hydrocarbons: the reaction temperature is 250° C., the reaction pressure is 1.5 MPa, the space velocity of the entrance raw materials syngas is 4.0 NL/g-cat/h and the ratio of H₂/CO of the entrance raw materials syngas is 0.67.

The property parameters of the catalyst for Fischer-Tropsch synthesis reaction prepared from the above mentioned examples are listed in the following Table 1. As can be seen from Table 1, the catalyst according to the present invention in the slurry bed reactor is operated at a high reaction space velocity, and maintained a very high reaction activity of Fischer-Tropsch synthesis, wherein both the conversion rates of CO and H₂ are 80% or more, the effective hydrocarbons selectivity (C₂=−C₄=+C₅⁺) is remained at 90 wt % or more, the methane selectivity is 3.0% or less, the olefin selectivity of C₅=−C₁₁=decreased to 60 wt % or less, and the yield (oil and wax) is very high with both of them more than 0.45 g/g-cat./h. Therefore, the catalyst according to the present invention is especially suitable for synthesizing diesel, gasoline, wax and other products from syngas in the slurry bed reactor.

TABLE 1
Catalyst evaluation results

| | Catalyst | | |
|---|---|---|---|
| | Catalyst A | Catalyst B | Catalyst C |
| CO conversion rate, % | 91.2 | 85.7 | 86.0 |
| H₂ conversion rate, % | 82.0 | 80.3 | 81.8 |
| Hydrocarbons selectivity, wt % | | | |
| CH₄ | 3.00 | 2.31 | 2.95 |
| C₂-C₄ | 11.23 | 8.95 | 10.23 |
| C₅+ | 85.77 | 88.74 | 86.92 |
| C₂⁼-C₄⁼ + C₅⁺ | 90.05 | 93.00 | 91.39 |
| C₅⁼-C₁₁⁼/C₅-C₁₁, % | 60.34 | 58.15 | 57.17 |
| CO₂ selectivity, mol % | 24.53 | 20.98 | 22.64 |
| yield (oil and wax), g/g-cat./h | 0.56 | 0.42 | 0.60 |

The present invention has been described above in detail. For the skilled in the art, it is obvious that many improvements and modifications can be made without deviating from the spirit of the present invention. All of these modifications and improvements are included in the protection scope of the present invention.

1. A Fe-based catalyst for Fischer-Tropsch synthesis including Fe as its main component having a form of a complete oxide thereof, the catalyst comprising:
an oxide of IB group metal Cu and/or Ag as a reducing promoter;
at least one metal oxide of IA group metal M as an electron promoter, wherein the IA group metal M is selected from the group consisting essentially of Li, Na, K and Rb;
at least one VIII group noble metal M′ as a hydrogenation promoter, wherein the VIII group noble metal M′ is selected from the group consisting essentially of Ru, Rh, Pd and Pt; and
SiO₂ as a structure promoter;
wherein an as-finished Fe content of the catalyst is between approximately 30 wt %-70 wt %.

2. The Fe-based catalyst according to claim 1, wherein the as-finished Fe content is between approximately 45 wt %-60 wt %.

3. The Fe-based catalyst according to claim 1, wherein a weight parts ratio of each component is approximately:
Fe:Cu:Ag:the electron promoter:the hydrogenation promoter:the structure promoter:100:0-20:0-10:0.05-15:0.001-5:0.1-50, wherein a content of each metal component is calculated based on a metal element of the metal component, and a content of the structure promoter is calculated based on an oxide of the structure promotor; a contents of a content of Cu and Ag not being zero simultaneously.

4. The Fe-based catalyst according to claim 2, wherein a weight parts ratio of each component is approximately:Fe:Cu:Ag:the electron promoter:the hydrogenation promoter:the structure promoter=100:0-20:0-10:0.05-15: 0.001-5:0.1-50, wherein a content of each metal component is calculated based on a metal element of the metal component, and a content of the structure promoter is calculated based on an oxide of the structure promotor; a content of Cu and Ag not being zero simultaneously.

5. The Fe-based catalyst according to claim 3, wherein a weight parts ratio of each component is approximately: Fe:Cu:Ag:the electron promoter:the hydrogenation promoter:the structure promoter=100:0-8:0-2:0.5-8:0.01-0.5:5-35, wherein a content of each metal component is calculated based on a metal element of the metal component, and a content of the structure promoter is calculated based on an oxide of the structure promotor; a content of Cu and Ag not being zero simultaneously.

6. The Fe-based catalyst according to claim 4, wherein a weight parts ratio of each component is approximately:Fe:Cu:Ag:the electron promoter:the hydrogenation promoter:the structure promoter=100:0-8:0-2:0.5-8:0.01-0.5: 5-35, wherein a content of each metal component is calculated based on a metal element of the metal component, and a content of the structure promoter is calculated based on an oxide of the structure promotor; a content of Cu and Ag not being zero simultaneously.

7. The Fe-based catalyst according to claim 3, wherein the IA group metal M is K or Li, and/or the VIII group noble metal M′ is Ru or Pt.

8. The Fe-based catalyst according to claim 4, wherein the IA group metal M is K or Li, and/or the VIII group noble metal M′ is Ru or Pt.

9. The Fe-based catalyst according to claim 7, wherein components of the catalyst are Fe, Cu, Li, Ru and SiO₂; Fe, Ag, K, Pt and SiO₂; or Fe, Cu, K, Pt and SiO₂.

10. The Fe-based catalyst according to claim 8, wherein components of the catalyst are Fe, Cu, Li, Ru and SiO₂; Fe, Ag, K, Pt and SiO₂; or Fe, Cu, K, Pt and SiO₂.

11. A preparation method of the Fe-based catalyst of claim 1, the method comprising the steps of:
(1) preparing a solution of Fe salt;
(2) co-precipitating the solution of Fe salt with an alkaline compound as a precipitant to obtain a precipitate;
(3) washing the precipitate;
(4) pulping the precipitate;
5) adding a solution of IB group metal Cu and/or Ag salt, a solution of IA group metal M salt and a structure promoter SiO₂ to obtain a mixed slurry, wherein the structure promoter SiO₂ is silica sol or a silicate of IA group metal M; and
(6) spray-drying the mixed slurry to obtain a molded catalyst, (7) isometric impregnating the molded catalyst in a solution of at least one kind of VIII group noble metal M′ salt and
(7) drying and roasting to obtain the catalyst.

12. The preparation method of the Fe-based catalyst according to claim 11, wherein:
the solution of Fe salt in the step (1) is a ferric nitrate solution or a ferric sulfate solution with a concentration of approximately 0.5-10 mol/L;
the precipitant of alkaline compound used in the step (2) is selected from the group consisting essentially of Na₂CO₃, ammonia and (NH₄)₂CO₃, the concentration of an aqueous solution of the alkaline precipitant is approximately 1-6 mol/L;
the solution of Cu salt used in the step (5) is a cupric nitrate solution or a cupric sulfate solution with a concentration of approximately 1-4 mol/L;
the solution of Ag salt used in the step (5) is a silver nitrate solution with a concentration of approximately 0.1-3 mol/L;
the solution of IA group metal M salt used in the step (5) is a solution of carbonate or acetate of IA group metal M with a concentration of approximately 0.5-25 wt %;
a concentration of SiO₂ in the solution of the structure promoter used in the step (5) is approximately 5-50 wt %; and/or
a solution of VIII group noble metal M′ salt used in the step (6) is a nitrate solution.

13. The preparation method of the Fe-based catalyst according to claim 12, wherein a precipitation temperature in the step (2) is approximately 40-90° C., and a pH of the solution is approximately 6-10; and a precipitation time is approximately 10-30 min.

14. The preparation method of the Fe-based catalyst according to claim 13, wherein SiO₂ and IA group metal M added into the slurry of the precipitate in the step (5) are in each in a form of a silicate solution of IA group metal M prepared in advance according to the required proportion, the SiO₂/M₂O in the solution having a molar ration of approximately 1-10.

15. The preparation method of the Fe-based catalyst according to claim 14, wherein:
a drying temperature in the step (7) is approximately 60-120° C.; and
a roasting temperature in the step (7) is approximately 200-600° C.

16. The preparation method of the Fe-based catalyst according to claim 11, the method including the steps of:
(1) preparing a solution of ferric nitrate or ferric sulfate with a concentration of approximately 1-5 mol/L;
(2) preparing an aqueous solution of an alkaline precipitant with a concentration of approximately 1.5-4.5 mol/L;
(3) precipitating by coflowing the solutions of (1) and (2) at a temperature of approximately 55-85° C. to obtain a slurry of a precipitate with pH of 6-9 and a precipitation time of 10-30 min;
(4) aging the slurry of the precipitate obtained;
(5) vacuum filtering the precipitate;
(6) washing the precipitate to obtain a washed precipitate;
(7) adding water to the washed precipitate;
(8) pulping the washed precipitate;
(9) adding a solution of Cu salt with a concentration of approximately 1.5-3.0 mol/L and/or a solution of Ag salt with a concentration of approximately 0.5-1.5 mol/L, a silica sol or a silicate of IA group metal M with a concentration of SiO₂ of approximately 15-40 wt % and a solution of IA group metal M salt with a concentration of approximately 10-20 wt % to the washed precipitate;
(10) stirring the washed precipitate to obtain a catalyst slurry;
(11) spray-drying the catalyst slurry; and
(12) selecting particles of approximately 50-100 μm;
(13) isometric impregnating the particles in a solution of VIII group noble metal M′ salt into the slurry; and
(14) drying and roasting the slurry to obtain the finished catalyst.

17. The preparation method of the Fe-based catalyst according to claim 12, the method comprising the steps of:
(1) preparing a solution of ferric nitrate or ferric sulfate with a concentration of approximately 1-5 mol/L;
(2) preparing an aqueous solution of an alkaline precipitant with a concentration of approximately 1.5-4.5 mol/L;
(3) precipitating by coflowing the solutions of (1) and (2) at a temperature of approximately approximately 55-85° C. to obtain a slurry of a precipitate with pH of approximately 6-9 and a precipitation time of approximately 10-30 min;
(4) aging the slurry of the precipitate obtained;
(5) vacuum filtering the precipitate;
(6) washing the precipitate to obtain a washed precipitate;
(7) adding water to the washed precipitate;
(8) pulping the washed precipitate;
(9) adding a solution of Cu salt with a concentration of approximately 1.5-3.0 mol/L and/or a solution of Ag salt with a concentration of approximately 0.5-1.5 mol/L, a silica sol or a silicate of IA group metal M with a concentration of SiO₂ of approximately 15-40 wt % and a solution of IA group metal M salt with a concentration of 10-20 wt % to the washed precipitate;
(10) stirring the washed precipitate to obtain a catalyst slurry;
(11) spray-drying the catalyst slurry;
(12) selecting particles of approximately 50-100 μm;
(13) isometric impregnating the particles in a solution of VIII group noble metal M′ salt into the slurry; and
(14) drying and roasting the slurry to obtain the finished catalyst.

18. A method for producing hydrocarbons by a low-temperature Fischer-Tropsch synthesis reaction comprising:
catalyzing a reaction using the Fe-based catalyst according to claim 1.

19. The method according to claim 18, further comprising:
pretreating the Fe-based catalyst in a slurry bed reactor with by cycling a tail gas; and
conducting the Fischer-Tropsch synthesis reaction in the presence of the pretreated catalyst.

20. The method according to claim 19, wherein the step of pretreating comprises:
mixing the catalyst and a molten Fiseher-Tropsch wax well into a slurry; loading the slurry into a slurry bed reactor with the cycled tail gas; purging the slurry with an inactive gas; importing a reducing gas;
adjusting the pressure of the reactor to a reduction pressure of approximately 0.1-5 MPa, a space velocity of the reducing gas being approximately 0.5-5.0 NL/g-cat/h;
gradually warming the reactor to a reduction temperature of approximately 180-300° C., and reducing for approximately 2-48 h; and
importing an inactive gas-containing reducing gas,
wherein a volume percentage of the inactive gas in the reducing gas is approximately 1%-20%, and the remaining is syngas with a ratio of H₂/CO of approximately 0.5-40.

21. The method according to claim 20, wherein the reducing gas is substantially pure H₂ or substantially pure CO or syngas,
when the reducing gas is a syngas, the hydrogen-to-carbon ratio of the syngas is approximately 0.01-99; the inactive gas is N₂ or Ar; the volume percentage of the inactive gas in the reducing gas is approximately 5%-15%; and/or a reduction temperature of the pretreatment process is approximately 210-280°, the reduction pressure is approximately 0.25-4 MPa, and the space velocity of the reducing gas is approximately 1.0-4.0 NL/g-cat/h.
